(19) United States
(12) Patent Application Publication
(10) Pub. No.: US 2026/0315800 A1
(43) Pub. Date: Oct. 15, 2026

(54) NEUROMODULATION/NEUROSTIMULATION SYSTEM FOR CONTROLLING A RESPIRATORY FUNCTION

(71) Applicants: ONWARD MEDICAL N.V., Eindhoven (NL); École Polytechnique Fédérale de Lausanne, Lausanne (CH); Centre Hospitalier Universitaire Vaudois, Lausanne (CH)

(72) Inventors: Jocelyne BLOCH, Paudex (CH); Grégoire COURTINE, Lausanne (CH); Robin DEMESMAEKER, Morges (CH); Leonie ASBOTH, Lausanne (CH); Nicolas HANKOV, Lausanne (CH); Markus RIEGER, Amsterdam (NL); Miroslav CABAN, Renens (CH); Francesco ACQUATI, Lausanne (CH); Vincent DELATTRE, Lausanne (CH)

(21) Appl. No.: 19/476,595

(22) PCT Filed: Apr. 17, 2024
(86) PCT No.: PCT/EP2024/060461

§ 371 Date: Oct. 17, 2025

Related U.S. Application Data
(60) Provisional application No. 63/496,932, filed on Apr. 18, 2023.

(30) Foreign Application Priority Data
Apr. 18, 2023 (EP) 23168611.4

(51) Int. Cl. A61N 1/36; A61N 1/05

(52) U.S. Cl. CPC A61N 1/3611; A61N 1/0551; A61N 1/3601; A61N 1/36135; A61N 1/36171; A61N 1/36178

(57) ABSTRACT

The present invention relates to a neuromodulation/neurostimulation system for supporting and/or controlling a function of the respiratory system of a patient, said function comprising a plurality of phases each involving one or more muscles. The system comprises:
at least one control unit, configured and/or adapted to provide stimulation data for each phase of said function of the respiratory system, and
at least one stimulation unit, operatively connected to the at least one control unit,
   wherein the at least one stimulation unit is configured and/or adapted to deliver electrical stimulation at least partially targeted to the dorsal roots of the thoracic region of the spinal cord innervating the trunk muscles of said patient,
   wherein said stimulation data include at least one stimulation parameter defined for each phase of said function of the respiratory system, and
   wherein, during each phase of said function of the respiratory system, the at least one control unit is configured and/or adapted to control the at least one stimulation unit to deliver electrical stimulation according to the at least one stimulation parameter defined for the ongoing phase of said function.

### TECHNICAL FIELD

The present invention belongs to the technical field of neuromodulation/neurostimulation for the improvement of an autonomic function of a patient, more specifically a function of the respiratory system such as breathing or coughing.

In particular, the present invention relates to a neuromodulation/neurostimulation system for supporting and/or controlling a function of the respiratory system of a patient.

### BACKGROUND

Preferably, said patient is a human with spinal cord injury (SCI) and/or other neurological disorders such as a stroke, multiple sclerosis, autonomic failure, autonomic neuropathy or cancer of the neurological tissue, which impair operation of descending sympathetic pathways that normally facilitate control of autonomic functions.

### SUMMARY

The spinal cord is an integral part of the central nervous system (CNS). SCI may result not only in motor and sensory deficits, but also in autonomic dysfunctions. In particular, SCI may result in disconnection of some, most, or all descending sympathetic pathways that carry signals responsible for triggering, e.g., a respiratory function such as a breathing or coughing.

Also, in order to carry out a respiratory function, such as breathing or coughing, it is necessary that the muscles involved in each phase of said function are sufficiently innervated.

Autonomic dysfunctions following SCI or other neurological disorders have a significant impact on everyday life of a patient and may represent potentially life-threatening conditions. For this reason, autonomic dysfunctions are ranked as a top healthcare priority, even more than restoration of motor functions, e.g., walking.

Disfunctions of the respiratory system may render the patient vulnerable to sever diseases and/or infections of the respiratory tract, which may require specific long-term treatment and/or hospitalization and significantly increase the mortality rate.

In particular, breathing disfunctions may lead to insufficient ventilation.

On the other hand, coughing disfunctions may be prejudicial to secretions removal, thereby increasing the risk of severe infections of the respiratory tract.

One current approach for supporting and/or controlling a function of the respiratory system, such as breathing or coughing, involves delivery of continuous Epidural Electrical Stimulation (EES).

An exemplary application of this approach is provided in DiMarco AF et al., Lower Thoracic Spinal Cord Stimulation to Restore Cough in Patients With Spinal Cord Injury: Results of a National Institutes of Health-Sponsored Clinical Trial. Part I: Methodology and Effectiveness of Expiratory Muscle Activation, Arch Phys Med Rehabil. 2009;90(5):717-725. doi:10.1016/j.apmr.2008.11.013, describing a clinical trial directed at evaluating capacity of lower thoracic spinal cord stimulation (SCS) to activate the expiratory muscles and generate large airway pressures and high peak airflows characteristic of cough, in subjects with tetraplegia.

The clinical trial revealed that lower thoracic SCS results in near maximal activation of the expiratory muscles with the consequent generation of high airway pressures and peak airflow rates, characteristic of a normal cough, thus facilitating removal of secretions, reducing the incidence of respiratory tract infections and atelectasis and associated morbidity and mortality in subjects with SCI.

Also, Hachmann, J. T. et al. Review of Epidural Spinal Cord Stimulation for Augmenting Cough after Spinal Cord Injury. Front. Hum. Neurosci. 11, 1-10 (2017) describes a clinical trial directed at investigating the effectiveness of EES of the lower thoracic spinal cord for restoring cough by evoking expiratory muscle contraction to generate large positive airway pressures and expulsive air flow.

The clinical trial (involving 9 SCI subjects) revealed that epidural SCS of the lower thoracic spinal cord holds promise for cough restoration in individuals with cervical SCI, evoking near-maximal expiratory cough performance with positive airway pressures and peak flow rates approximating physiological cough.

Also, a long-term follow-up of this initial clinical trial revealed a sustained efficacy of this approach in alleviating respiratory complications, thus potentially reducing medical intervention and healthcare costs, as well as the need for trained caregiver assistance.

Still further, Malone IG et al. Closed-Loop, Cervical, Epidural Stimulation Elicits Respiratory Neuroplasticity after Spinal Cord Injury in Freely Behaving Rats. eNeuro. 2022; 9(1): ENEURO. 0426-21.2021. Published 2022 Feb 9. doi:10.1523/ENEURO.0426-21.2021 describes a clinical trial on rats, directed at investigating the potential of closed-loop stimulation triggered via healthy hemidiaphragm (EMG) to elicit functional neuroplasticity in spinal respiratory pathways after cervical SCI (cSCI). To test this, closed-loop, electrical, epidural stimulation (CLES) was delivered at the level of the phrenic motor nucleus (C4) for 3 days after C2 hemisection (C2HS) in freely behaving rats.

The clinical trial revealed the potential of CLES in future therapy to address respiratory deficiencies associated with cSCI.

However, this clinical trial only involved delivery of electrical stimulation to the cervical region of the spinal cord, while stimulation to the thoracic region was not contemplated.

Stimulation of the thoracic region of the spinal cord plays an important role in supporting a respiratory function such as breathing or coughing.

Also, by stimulating the thoracic region of the spinal cord, it is possible to support an upright posture of the spinal cord, which relieves resistance to the diaphragm and the rib cage, thereby allowing to perform a function of the respiratory system, e.g. breathing or coughing, with increased effectiveness and reduced effort.

Still further, this experiment was carried out with respect to a population of rats with spinal cord hemisection, where only the left side of the spinal cord was injured. In this context, electrical stimulation was triggered by spare muscle activity in the right side of the diaphragm (contralesional). However, this approach would not be possible in case of traumatic SCI affecting not solely one side of the spinal cord and/or in case of complete SCI.

Functions of the respiratory system usually include a plurality of phases, each involving specific muscles.

For instance, breathing is a multi-phase function composed of an active inhalation phase alternating with a passive expiration phase, each involving the activation of different muscles.

FIG. 1 provides a schematic illustration of the innervation map of the main muscles involved during a breathing function in a human subject.

The diaphragm, which is innervated at the cervical level, in particular at spinal cord level C3-C5, represents the primary muscle for a breathing function in a human subject (FIG. 1).

The intercostal and abdominal muscles, which are innervated at the thoracic level, in particular at spinal cord level T1-T11 (intercostal muscles) and T7-L1 (abdominal muscles), represent the main accessory muscles for a breathing function in a human subject (FIG. 1).

SCI and/or other neurological disorders may cause breathing deficiencies, which may lead to insufficient ventilation.

Coughing is a multi-phase function including three phases, namely an inhalation phase, a compressive phase and an expulsive phase.

The different phases and involved muscles for a coughing function in a human subject are illustrated in FIG. 2.

In particular, coughing includes an inhalation phase, a compressive phase and an expulsive phase, each involving specific muscles.

In particular, the inhalation phase involves the diaphragm, the external intercostal muscles, the scalene muscles, and the parasternal muscles (FIG. 2).

During the inhalation phase, the glottis is open.

The compressive phase involves the abdominal muscles, the oblique muscles, and the internal intercostal muscles (FIG. 2).

During the compressive phase, the glottis is closed.

Finally, the expulsion phase involves the abdominal muscles, the oblique muscles, and the internal intercostal muscles (FIG. 2).

During the expulsion phase, the glottis is again open.

The same spinal cord regions mentioned above with respect to breathing, i.e. C3-C5, T1-T11 and T7-L1, are as well involved during coughing.

SCI and/or other neurological disorders may lead to insufficient coughing, which hinders the ability of the patient to remove secretions. As mentioned, this may cause potentially life-threatening infections of the respiratory tract.

Continuous activation of the respiratory muscles through delivery of continuous EES as described above has proven unable to provide an effective support for functions of the respiratory system such as breathing or coughing, which are multi-phase functions each phase involving the activation of different muscles.

Otherwise stated, continuous EES does not allow to support a function of the respiratory system, e.g. breathing or coughing, in a phase-specific manner.

Another known approach for supporting and/or controlling a respiratory function in a patient with SCI or other neurological disorders involves delivery of Functional Electrical Stimulation (FES) to the abdominal muscles.

Here, electrical stimulation is delivered to the abdominal muscles in order to provoke direct activation of said muscles.

For instance, Gollee H et al. A control system for automatic electrical stimulation of abdominal muscles to assist respiratory function in tetraplegia. Med Eng Phys. 2007 Sep; 29(7): 799-807. doi: 10.1016/j. medengphy.2006.08.007. Epub 2006 Oct 10. PMID: 17035064 describes a control system configured to automatically trigger abdominal muscle stimulation, synchronized with a subject's voluntary respiratory activity (FIG. 3). In addition, referring to FIG. 3, an algorithm (e.g. so-called Simulink algorithm) for FES stimulation can use a closed loop arrangement with a spirometer. Four stimulation channels can be used on both sides: two for rectus abdominis; two for lateral abdominal muscles (transversi +obliqui ext et int). A coughing algorithm can be implemented, e.g. by checking: the flow being low (compared to a threshold); and/or going towards zero (expiration). A breathing algorithm can be implemented, e.g. by using cross correlation to detect breathing vs. speaking.

In particular, the system was tested in four subjects with a functionally complete lesion at level C4-C6, aged between 16 and 46 years, 3 months to 5 years post injury, who were breathing spontaneously. The system was implemented with an algorithm configured to deliver automatic stimulation patterns, detecting cough and quiet breathing while suppressing stimulation during other activities such as speaking. That is, a closed-loop is controlled by measuring air flow.

Also, the system supports a quiet breathing stimulation that provides stimulation only during the expiration phase of each breathing cycle. That is, no support is provided during the inhalation phase, which might be useful especially in case of a severe breathing deficiencies.

The experiment revealed marked increases in V(T) (between 9% and 71% of baseline) and CPF (between 31% and 54% of baseline), suggesting that the technique may have potential use in both acute and established tetraplegia to increase minute ventilation and to improve cough clearance of secretions.

Although effective, these known approaches still have some drawbacks, in particular in terms of reduced effectiveness in supporting and/or controlling a function of the respiratory system such as breathing or coughing.

Also, these known approaches have often proven unable to properly mimic the natural dynamics of the supported function, such as breathing or coughing.

That is, the patient may have a feeling which is still far from natural.

Therefore, there is a need for an improved solution that allows to support a function of the respiratory system, e.g. breathing or coughing, with improved effectiveness and further in a manner that is perceived close to natural by the patient.

It is an object of the present invention to provide a neuromodulation/neurostimulation system capable of supporting and/or controlling a function of the respiratory system, in particular a breathing function or a coughing function, with improved effectiveness and in a manner that is perceived close to natural by the patient.

According to the invention, a neuromodulation/neurostimulation system for supporting and/or controlling a function of the respiratory system of a patient, said function comprising a plurality of phases each involving one or more muscles, comprises:
at least one control unit, configured and/or adapted to provide stimulation data for at least a first phase and a second phase (optionally each phase) of said function of the respiratory system, and
at least one stimulation unit, operatively connected to the at least one control unit,
   wherein the at least one stimulation unit is configured and/or adapted to deliver electrical stimulation at least partially targeted to the dorsal roots of the thoracic region of the spinal cord innervating the trunk muscles of said patient,
   wherein said stimulation data include at least one stimulation parameter defined for the at least first phase and second phase (optionally each phase) of said function of the respiratory system, and
   wherein, during the at least first phase and second phase (optionally each phase) of said function of the respiratory system, the at least one control unit is configured and/or adapted to control the at least one stimulation unit to deliver electrical stimulation according to the at least one stimulation parameter defined for the ongoing and/or respective phase of said function.

The present invention provides neuromodulation/neurostimulation system.

In particular, the system is configured and/or adapted for supporting and/or controlling a function of the respiratory system of a patient, said function comprising a plurality of phases each involving one or more muscles.

Preferably, said patient is a human affected by spinal cord injury (SCI) and/or other neurological disorders such as a stroke, multiple sclerosis, autonomic failure, autonomic neuropathy or cancer of neurological tissue.

The system comprises at least one control unit configured and/or adapted to provide stimulation data for at least first and second phases (optionally each phase) of said function of the respiratory system.

For instance, the system may include a single control unit.

The system also includes at least one stimulation unit, operatively connected to the at least one control unit.

For instance, the system may include a single stimulation unit.

The stimulation unit may include at least one implantable pulse generator (IPG).

Also, the stimulation unit may include at least one electrode.

The stimulation unit is configured and/or adapted to deliver electrical stimulation at least partially targeted to the dorsal roots of the thoracic region of the spinal cord innervating the trunk muscles of the patient.

In particular, said stimulation data include at least one stimulation parameter defined for the first phase and the second phase (optionally each phase) of said function of the respiratory system.

During the first phase and the second phase (optionally each phase) of said function of the respiratory system, the control unit is configured and/or adapted to control the stimulation unit to deliver electrical stimulation according to the at least one stimulation parameter defined for the ongoing and/or respective phase of said function.

Therefore, said function of the respiratory system can be controlled and/or supported in a phase-specific manner.

That is, the muscles that are involved in each phase of said function of the respiratory system can be activated at the right time and according to stimulation parameter(s) that are specifically defined for the ongoing phase of said function.

The invention is based on the basic idea that, by providing a phase-specific electrical stimulation, a respiratory system function, such as breathing or coughing, can be supported and/or controlled with improved effectiveness and in a manner which is perceived as natural, or at least close to natural, by the patient. For instance, improved ventilation and/or stronger coughing can be obtained.

Also, electrical stimulation of the dorsal roots innervating the trunk muscles allows to support an upright posture of the spinal cord, which relieves resistance to the diaphragm and the rib cage, such that the patient is enabled to perform a respiratory system function with enhanced effectiveness and reduced effort.

In particular, supporting an upright posture of the spinal cord during a breathing function allows to facilitate physiological ventilation of the lungs and improve respiration volumes.

Also, an upright posture of the spinal cord may facilitate secretions expulsion during coughing.

The at least one stimulation unit may be configured and/or adapted to deliver electrical stimulation targeted to the dorsal roots of the low thoracic region and/or the upper thoracic region of the spinal cord.

In particular, electrical stimulation targeted to the dorsal roots of the upper thoracic region of the spinal cord may support the inhalation phase during either breathing or coughing, while stimulation targeted to the dorsal roots of the lower thoracic region may support the expiration phase during breathing or the expulsion phase during coughing.

The at least one stimulation unit may be further configured and/or adapted to deliver electrical stimulation targeted to the dorsal roots of the cervical region of the spinal cord.

In particular, electrical stimulation targeted to the dorsal roots of the cervical region of the spinal cord may support the inhalation phase during either breathing or coughing.

In particular, the at least one stimulation unit may be configured and/or adapted to deliver electrical stimulation targeted to the dorsal roots at spinal cord level C2-C4, for controlling the diaphragm.

Additionally or alternatively, the at least one stimulation unit may be configured and/or adapted to deliver electrical stimulation targeted to the dorsal roots at spinal cord level T1-T12, for controlling the intercostal muscles.

Additionally or alternatively, the at least one stimulation unit may be configured and/or adapted to deliver electrical stimulation targeted to the dorsal roots at spinal cord level T7-L1, for controlling the abdominal and oblique muscles.

In particular, the at least one stimulation unit may be configured and/or adapted to deliver Epidural Electrical Stimulation (EES).

Preferably, the at least one stimulation unit is configured and/or adapted to deliver multi-site EES.

This allows to obtain a more effective activation of the involved muscles.

Also, the at least one stimulation unit may be configured and/or adapted to deliver Transcutaneous Spinal Cord Stimulation (tSCS).

Preferably, the at least one stimulation unit may be configured and/or adapted to deliver multi-site tSCS.

Even more preferably, the at least one stimulation unit may include four independent stimulation channels (optionally more) which are configured and/or adapted to deliver multi-site tSCS.

As mentioned, multi-site stimulation allows to obtain a more effective activation of the involved muscles.

As a further alternative, the at least one stimulation unit may be configured and/or adapted to deliver EES in combination with at least one among tSCS, Functional Electrical Stimulation (FES), Peripheric Nerve Stimulation (PNS), Vagus Nerve Stimulation (VNS), Neuromuscular Electrical Stimulation (NMES), and/or Diaphragm pacing/pacemakers.

Preferably, the at least one stimulation unit may be configured and/or adapted to deliver EES in combination tSCS.

By delivering combined EES and tSCS stimulation, it is possible to further improve effectiveness in supporting said function of the respiratory system, e.g. breathing or coughing.

Even more preferably, the at least one stimulation unit may be configured and/or adapted to deliver multi-site EES in combination with multi-site tSCS.

As mentioned, multi-site stimulation allows to obtain a more effective activation of the involved muscles.

The at least one control unit may be further configured and/or adapted to control the at least one stimulation unit to deliver electrical stimulation at least partially targeted to the dorsal roots of the thoracic region of the spinal cord innervating the trunk muscles for blood pressure control.

The delivery of electrical stimulation for controlling both a function of the respiratory system and blood pressure in a patient with SCI and/or other neurological disorders may be particularly beneficial during an acute phase, e.g. the first weeks after injury, where patients under spinal shock are often subject severe orthostatic hypotension and equipped with ventilatory assistance.

In one embodiment, the system may be an open-loop system.

Here, the system optionally further comprises a feedback module.

In particular, the feedback module is configured and/or adapted to provide a feedback (e.g. an indication) to the patient on a transition from one phase to the subsequent phase of said function of the respiratory system.

Accordingly, the patient is enabled to synchronize his/her actions according to the received feedback from the feedback module.

Here, the system may further comprise a mobile device, e.g. a smartphone, a smartwatch or the like, including display means allowing the patient to provide a user input to trigger a function of the respiratory system, in particular a breathing function or a coughing function, based on the feedback from the feedback module. Advantageously, said feedback may include an acoustic feedback.

Alternatively, said feedback may include one or more external inputs.

In another embodiment, the system may be a closed-loop system.

Here, the system further comprises at least one sensor, operatively connected to the at least one control unit.

For instance, the system may include a single sensor.

In particular, said at least one sensor is configured and/or adapted to detect a transition from one phase to the subsequent phase of said function of the respiratory system.

Also, the at least one sensor is configured and/or adapted to transmit data regarding a detected phase transition to the at least one control unit, in real time.

Based on the data received from the at least one sensor, the at least one control unit is configured and/or adapted to control the at least one stimulation unit to deliver targeted stimulation for controlling the involved one or more muscles for the ongoing phase of said function of the respiratory system, according to at least one predefined stimulation parameter for said phase.

Real-time as used here as a term is to be understood such that real-time is is a guaranteed level of computer responsiveness within a specified time constraint, usually milliseconds or microseconds, between an event and its response deadline. Real-time describes a human sense of time (rather than machine time) that seems immediate, see also DIN ISO/IEC 2382 and DIN 44300.

Said function of the respiratory system may be a coughing function, in particular including:
an inhalation phase;
a compressive phase, and
an expulsive phase.

In one embodiment, the system can be an open-loop system for supporting and/or controlling a coughing function.

Here, the system further includes a feedback module.

The feedback module is configured and/or adapted to provide a feedback (e.g. an indication) to the patient on a transition from the inhalation phase to the compressive phase, and from the compressive phase to the expulsive phase.

Accordingly, the patient is enabled to synchronize his/her actions according to the received feedback from the feedback module.

Here the system may further comprise a mobile device (e.g. a smartphone, a smartwatch or the like) including display means allowing the patient to provide a user input to trigger a coughing function based on the feedback from the feedback module. Advantageously, said feedback may include an acoustic feedback.

Alternatively, said feedback may include one or more external inputs.

In another embodiment, the system can be a closed-loop system for supporting and/or controlling a coughing function.

Here, the system further comprises at least one sensor, operatively connected to the at least one control unit.

For instance, the system may include a single sensor.

The at least one sensor is configured and/or adapted to detect a transition from the inhalation phase to the compressive phase, and from the compressive phase to the expulsive phase.

Also, the at least one sensor is configured and/or adapted to transmit data regarding a detected phase transition to the at least one control unit, in real time.

Based on the data received from the at least one sensor, the at least one control unit is configured and/or adapted to control the at least one stimulation unit to:
during the inhalation phase, deliver stimulation to control the diaphragm and/or the external intercostal muscles and/or the scalene muscles and/or the parasternal muscles, according to at least one predefined stimulation parameter for said inhalation phase;
during the compressive phase, deliver stimulation to control the abdominal muscles and/or the oblique muscles and/or the internal intercostal muscles, according to at least one predefined stimulation parameter for said compressive phase, and
during the expulsive phase, deliver stimulation to control the abdominal muscles and/or the oblique muscles and/or the internal intercostal muscles, according to at least one predefined stimulation parameter for said expulsive phase.

The invention also provides an alternative embodiment relating to a closed-loop system for supporting and/or controlling a coughing function.

Here, the system further comprises at least one sensor, operatively connected to the at least one control unit.

For instance, the system may include a single sensor.

The at least one sensor is configured and/or adapted to detect a transition from the inhalation phase to the compressive phase, and from the compressive phase to the expulsive phase.

Also, the at least one sensor is configured and/or adapted to transmit data regarding a detected phase transition to the at least one control unit, in real time.

Based on the data received from the at least one sensor, the at least one control unit is configured and/or adapted to control the at least one stimulation unit to:

during the inhalation phase, deliver stimulation to the cervical region of the spinal cord of the patient according to at least one predefined stimulation parameter for said inhalation phase, to control diaphragm activity, and
during the compressive phase and the expulsive phase, deliver stimulation to the thoracic region of the spinal cord of the patient according to at least one predefined stimulation parameter for said compressive phase and at least one predefined stimulation parameter for said expulsive phase, to control compression and expulsion.

Sensors that can be implemented in closed-loop systems according to the invention may include one or more among:
a spirometer device;
a transcutaneous electromyography (EMG) sensor;
an implantable EMG sensor;
a photoplethysmography (PPG) sensor, and/or
a wearable strain sensor.

Said function of the respiratory system may be a breathing function, in particular including:
an inhalation phase, and
an expiration phase.

According to one embodiment, the system can be a closed-loop system for supporting and/or controlling a breathing function.

Here, the system further comprises at least one sensor, operatively connected to the at least one control unit.

For instance, the system may include a single sensor.

The at least one sensor is configured and/or adapted to detect a diaphragm activity of the patient.

Also, the at least one sensor is configured and/or adapted to transmit data on the detected diaphragm activity to the at least one control unit in real time.

Preferably, said at least one sensor includes at least one implantable EEG sensor.

For instance, said at least one sensor may include a single implantable EEG sensor.

The at least one control unit is configured and/or adapted to control the at least one stimulation unit to deliver stimulation based on the detected diaphragm activity of the patient.

By delivering electrical stimulation during both the inhalation phase and the expiration phase, it is possible to provide an improved support to the breathing function, allowing to achieve complete exhalation, which supports the physiological mechanism of breathing and improves ventilation.

In principle, a full exhalation triggers a succeeding strong and deep inhalation.

By improving ventilation, the occurrence of infections and/or diseases of the respiratory tract can be largely prevented.

According to some embodiments of the invention, the at least one stimulation parameter may include at least one among stimulation frequency, stimulation amplitude, pulse width and/or carrier frequency. For example, such parameters may relate to signal characteristics.

In particular, stimulation can be delivered with stimulation frequency between 10 Hz and 10k Hz.

In particular, stimulation can be delivered with stimulation amplitude between 0 mA and 250 mA or 0 V and 25 V.

In particular, stimulation can be delivered with pulse width between 1 μs and 500 μs.

In particular, EES can be delivered with stimulation frequency between 0.2 Hz and 100 Hz, stimulation amplitude between 0 mA and 250 mA, pulse width between 1 ms and 0.5 ms, and carrier frequency between 5 khZ and 10khZ.

In particular, tSCS can be delivered with stimulation frequency between 1 Hz and 606 Hz, stimulation amplitude between 0 mA and 20 mA, and pulse width of 300 μs.

Additionally or alternatively to any of the above, the at least one stimulation parameter may include a delivery parameter or characteristic. For example, the delivery parameter may refer to respective region(s) of the spinal cord to which stimulation is to be applied during the different phases; and/or identity of a respective electrode(s) used to deliver the stimulation during the different phases.

The neuromodulation/neurostimulation system according to the invention is suitable for use in a method for supporting and/or controlling a function of the respiratory system, e.g. breathing or coughing, in a patient with SCI and/or other neurological disorders. In any of the foregoing, the at least one stimulation parameter may comprise a parameter that is different during the first phase than during the second phase, optionally a signal parameter. Additionally or alternatively, the at least one stimulation parameter may comprise a parameter that switches value at a transition between the first phase and the second phase, optionally a signal parameter that switches value. Additionally or alternatively, the at least one stimulation parameter may comprises a parameter that transitions progressively between the first phase and the second phase, optionally a signal parameter that transitions progressively.

In any of the foregoing, the at least one parameter may comprise a signal frequency, the signal frequency during the first phase being different from the signal frequency during the second phase. For example, the signal frequency during an inhalation phase may be lower than the signal frequency during an expiration phase. For example: the frequency during the inhalation phase may be about half of the frequency during the expiration phase; and/or the frequency may transition between a first frequency value during the inhalation phase, and a second frequency value during the expiration phase, the first frequency value being about half of the second frequency value; and/or the frequency may transition progressively between the inhalation phase and the expiration phase; and/or the frequency may switch at a transition between the inhalation phase and the expiration phase; and/or the frequency during the inhalation phase may be within a range of 100 to 200 Hz (optionally about 120Hz), and the frequency during the expiration phase may be within a range of 150 to 400 Hz (optionally about 250 Hz).

In a closely related aspect, the invention provides a method of operating a neuromodulation and/or neurostimulation system for supporting and/or for controlling a function of the respiratory system of a patient, said function comprising a plurality of phases each involving one or more muscles, the method comprising: generating during (or for) a first phase of the respiratory cycle, a first stimulation signal having a first characteristic; and generating during (or for) a second phase of the respiratory cycle, a stimulation signal having a second characteristic at least partially different from the first characteristic.

In a closely related aspect, the invention provides a method of neuromodulation and/or neurostimulation for supporting and/or controlling a function of the respiratory system of a patient, said function comprising a plurality of phases each involving one or more muscles, the method comprising: delivering electrical stimulation to a region of the spinal cord during (or for) a first phase of the respiratory cycle, the electrical stimulation having a first characteristic; and delivering electrical stimulation to a region of the spinal cord during (or for) a second please of the respiratory cycle, the electrical stimulation having a second characteristic at least partially different from the first characteristic.

In either of these methods, the at least partial difference in the first and second characteristics may include a difference in signal characteristic, for example, a difference in one or more of: stimulation frequency; stimulation amplitude; pulse width; and/or carrier frequency. Additionally or alternatively, the at least partial difference in the first and second characteristics may include a difference in delivery characteristic, for example: respective region(s) of the spinal cord to which stimulation is to be applied during the different phases; and/or identity of one or more electrodes used to deliver the stimulation.

### BRIEF DESCRIPTION OF THE FIGURES

Further details and advantages of the present invention shall now be disclosed in connection with the drawings, where:

FIG. 1 is a diagram showing the innervation map of the main muscles involved during a breathing function in a human subject;

FIG. 2 is a diagram showing the inhalation phase, the compressive phase and the expulsive phase of a coughing function in a human subject, in particular illustrating air flow, intra-lung pressure, glottis status and main muscles involved during each phase;

FIG. 3 is a diagram showing a stimulation setup for controlling a coughing function as described in Gollee H et al., A control system for automatic electrical stimulation of abdominal muscles to assist respiratory function in tetraplegia, Cit.);

FIG. 4 is a block diagram showing a schematic overview of one embodiment of the neuromodulation/neurostimulation system according to the invention, wherein said system is an open-loop system configured to support and/or control a function of the respiratory system of a patient;

FIG. 5 is a block diagram showing a schematic overview of another embodiment of the neuromodulation/neurostimulation system according to the invention, wherein said system is a closed-loop system configured to support and/or control a function of the respiratory system of a patient;

FIG. 6 is a block diagram showing a schematic overview of yet another embodiment of the neuromodulation/neurostimulation system according to the invention, wherein said system is an open-loop system specifically configured to support and/or control a coughing function of a patient;

FIG. 7 is a block diagram showing a schematic overview of still another embodiment of the neuromodulation/neurostimulation system according to the invention, wherein said system is a closed-loop system specifically configured to support and/or control a coughing function of a patient;

FIG. 8 is a block diagram showing a schematic overview of a further embodiment of the neuromodulation/neurostimulation system according to the invention, wherein said system is a closed-loop system which is also specifically configured to support and/or control a coughing function of a patient;

FIG. 9 is a block diagram showing a schematic overview of a still further embodiment of the neuromodulation/neurostimulation system according to the invention, wherein said system is a closed-loop system which is specifically configured to support and/or control a breathing function of a patient;

FIGS. 10-11 are schematic diagrams referring to a clinical trial on human SCI patients, showing the positive effects of phase-specific EES in supporting a coughing function of the involved patients;

FIG. 12 is a schematic representation of a stimulation signal frequency characteristic that varies between first and second phases of a respiration function of a patient in some embodiments.

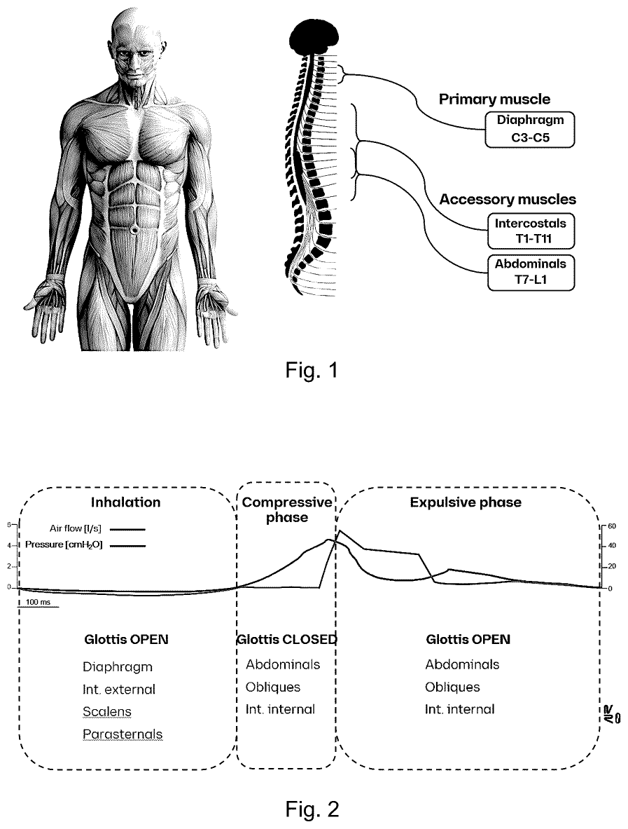

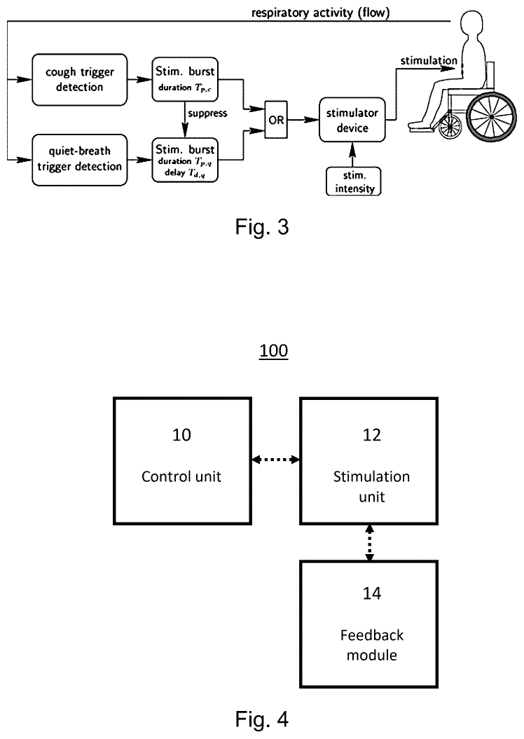

### DETAILED DESCRIPTION

The present invention provides a neuromodulation/neurostimulation system 100, 200, 300, 400, 500, 600 for supporting and/or controlling a function of the respiratory system in a patient, said respiratory function comprising a plurality of (e.g. two or three) phases, each involving one or more muscles.

In particular, the patient may be a human affected by spinal cord injury (SCI) and/or other neurological disorders such as a stroke, multiple sclerosis, autonomic failure, autonomic neuropathy or cancer of neurological tissue.

The system 100, 200, 300, 400, 500, 600 comprises at least one control unit 10, 20, 30, 40, 50, 60.

In the described embodiments (FIGS. 4-9), the system 100, 200, 300, 400, 500, 600 comprises a single control unit 10, 20, 30, 40, 50, 60.

The control unit 10, 20, 30, 40, 50, 60 is configured and/or adapted to provide stimulation data for each phase of said function of the respiratory system.

There is also at least one stimulation unit 12, 22, 32, 42, 52, 62, operatively connected to the control unit 10, 20, 30, 40, 50, 60.

In the described embodiments (FIGS. 4-9), the system 100, 200, 300, 400, 500, 600 comprises a single stimulation unit 12, 22, 32, 42, 52, 62.

The stimulation unit 12, 22, 32, 42, 52, 62 is configured and/or adapted to deliver electrical stimulation at least partially targeted to the dorsal roots of the thoracic region of the spinal cord innervating the trunk muscles of the patient.

Not shown is that the stimulation unit 12, 22, 32, 42, 52, 62 comprises at least one implantable pulse generator (IPG).

Also, not shown is that the stimulation unit 12, 22, 32, 42, 52, 62 comprises at least one electrode.

The stimulation data include at least one stimulation parameter defined for at least first and second phases (optionally for each phase) of said function of the respiratory system.

Here, said stimulation parameters include one or more of: stimulation frequency, stimulation amplitude, pulse width, and/or carrier frequency. Such parameters may be signal parameters or signal characteristics.

Additionally or alternatively, stimulation parameters can include delivery parameters or delivery characteristics. For example, the delivery parameter may refer to respective region(s) of the spinal cord to which stimulation is to be applied during the different phases; and/or identity of a respective electrode(s) used to deliver the stimulation during the different phases.

In particular, in the described embodiments, electrical stimulation is delivered with stimulation frequency between 10 Hz and 10 k Hz, stimulation amplitude between 0 mA and 250 mA or 0 V and 25 V and/or pulse width between 1 μs and 500 μs.

In particular, EES can be delivered with stimulation frequency between 0.2 Hz and 100Hz, stimulation amplitude between 0 mA and 250 mA, pulse width between 1 ms and 0.5 ms and carrier frequency between 5 khZ and 10 khZ.

In particular, tSCS can be delivered with stimulation frequency between 1 Hz and 606 Hz, stimulation amplitude between 0 mA and 20 mA, and pulse width of 300 μs.

During the at least first and second phases (optionally each phase) of said function of the respiratory system, the control unit 10, 20, 30, 40, 50, 60 is configured and/or adapted to control the at least one stimulation unit 12, 22, 32, 42, 52, 62 to deliver electrical stimulation according to the at least one stimulation parameter defined for the ongoing phase of said function.

Therefore, said function of the respiratory system can be controlled in a phase-specific manner.

Also, by at least partially stimulating the dorsal roots innervating the trunk muscles, it is possible to support an upright posture of the spinal cord, which relieves resistance to the diaphragm and the rib cage, which allows the patient to perform a respiratory system function with enhanced effectiveness and reduced effort.

In the described embodiments, the stimulation unit 12, 22, 32, 42, 52, 62 may be configured and/or adapted to deliver electrical stimulation targeted to the dorsal roots of the low thoracic region and/or the upper thoracic region of the spinal cord.

Alternatively, the stimulation unit 12, 22, 32, 42, 52, 62 may be configured and/or adapted to deliver electrical stimulation targeted to the dorsal roots of the cervical region of the spinal cord.

Preferably, the stimulation unit 12, 22, 32, 42, 52, 62 may be configured and/or adapted to deliver electrical stimulation targeted to the dorsal roots at spinal cord level:
C2-C4, for controlling the diaphragm, and/or
T1-T12, for controlling the intercostal muscles, and/or
T7-L1, for controlling the abdominal and oblique muscles.

In the described embodiments, the stimulation unit 12, 22, 32, 42, 52, 62 may be configured and/or adapted to deliver Epidural Electrical Stimulation (EES) Preferably, the stimulation unit 12, 22, 32, 42, 52, 62 is configured and/or adapted to deliver multi-site EES.

Alternatively, the stimulation unit 12, 22, 32, 42, 52, 62 may be configured and/or adapted to deliver Transcutaneous Spinal Cord Stimulation (tSCS).

Preferably, the stimulation unit 12, 22, 32, 42, 52, 62 is configured and/or adapted to deliver multi-site tSCS.

Advantageously, the stimulation unit 12, 22, 32, 42, 52, 62 may include four or more independent stimulation channels (not shown) which are configured and/or adapted to deliver multi-site tSCS.

Also, the stimulation unit 12, 22, 32, 42, 52, 62 may be configured and/or adapted to deliver EES in combination with tSCS.

Here, the stimulation unit 12, 22, 32, 42, 52, 62 is preferably configured and/or adapted to deliver multi-site EES in combination with multi-site tSCS.

Alternatively, EES may be delivered in combination with one among Functional Electrical Stimulation (FES), Peripheric Nerve Stimulation (PNS), Vagus Nerve Stimulation (VNS), Neuromuscular Electrical Stimulation (NMES), and/or Diaphragm pacing/pacemakers.

Said function of the respiratory system may be a coughing function, in particular including:
an inhalation phase;
a compressive phase, and
an expulsive phase.

Alternatively, said function of the respiratory system may be a breathing function, in particular including:
an inhalation phase, and
an expiration phase.

Advantageously, the at least one control unit 10, 20, 30, 40, 50, 60 may be further configured and/or adapted to control the at least one stimulation unit 12, 22, 32, 42, 52, 62 to deliver electrical stimulation at least partially targeted to the dorsal roots of the thoracic region of the spinal cord innervating the trunk muscles for blood pressure control.

As mentioned, the delivery of electrical stimulation for controlling both a function of the respiratory system and blood pressure in a patient with SCI and/or other neurological disorders may be particularly beneficial during an acute phase, e.g. the first weeks after injury.

In the following, embodiments of the neuromodulation/neurostimulation system according to the invention are described in connection with FIGS. 4-9.

FIG. 4 provides a schematic overview of a neuromodulation/neurostimulation system 100 according to an embodiment of the present invention.

Here, the system 100 is an open-loop system for supporting and/or controlling a function of the respiratory system in a patient with SCI and/or other neurological disorders.

The system 100 comprises a feedback module 14.

The feedback module 14 is configured and/or adapted to provide a feedback to the patient on a transition from one phase to the subsequent phase of said function of the respiratory system.

Accordingly, the patient may synchronize his/her actions according to the received feedback from the feedback module 14.

In particular, the system 100 may further comprise a mobile device (e.g., a smartphone, a smartwatch or the like) including display means allowing the patient to provide a user input to trigger a function of the respiratory system, in particular a breathing function or a coughing function, based on the feedback from the feedback module 14.

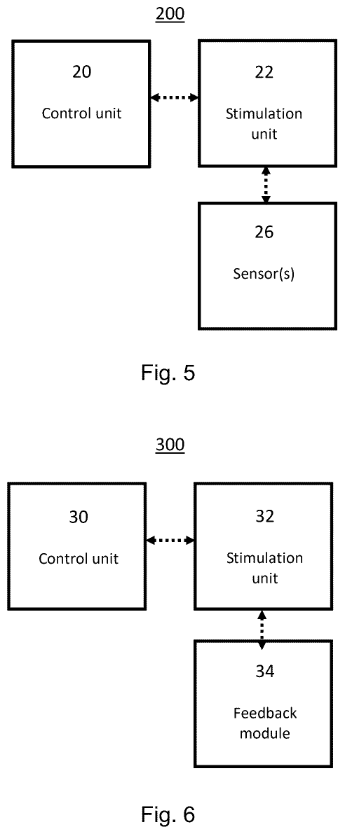

FIG. 5 provides a schematic overview of a neuromodulation/neurostimulation system 200 according to another embodiment of the present invention.

Here, the system 200 is a closed-loop system for supporting and/or controlling a function of the respiratory system in a patient with SCI and/or other neurological disorders.

The system 200 comprises at least one sensor 26, operatively connected to the at least one control unit 20.

For instance, the system 300 may include a single sensor 26.

The sensor 26 is configured and/or adapted to detect a transition from one phase to the subsequent phase of said function of the respiratory system, and transmit data regarding a detected phase transition to the at least one control unit 20, in real time.

Based on the data received from the at least one sensor 26, the at least one control unit 20 is configured and/or adapted to control the at least one stimulation unit 22 to deliver targeted stimulation for controlling the involved one or more muscles for the ongoing phase of said function of the respiratory system, according to at least one predefined stimulation parameter for said phase.

FIG. 6 provides a schematic overview of a neuromodulation/neurostimulation system 300 according to yet another embodiment of the present invention.

Here, the system 300 is an open-loop system for supporting and/or controlling a coughing function in a patient with SCI and/or other neurological disorders.

The system 300 includes a feedback module 34.

The feedback module 34 is configured and/or adapted to provide a feedback to the patient on a transition from the inhalation phase to the compressive phase, and from the compressive phase to the expulsive phase.

Accordingly, the patient may synchronize his/her actions according to the received feedback from the feedback module 34.

Here, the system 300 may further comprises a mobile device (e.g. a smartphone, a smartwatch or the like) including display means allowing the patient to provide a user input to trigger a coughing function based on the feedback from the feedback module 34.

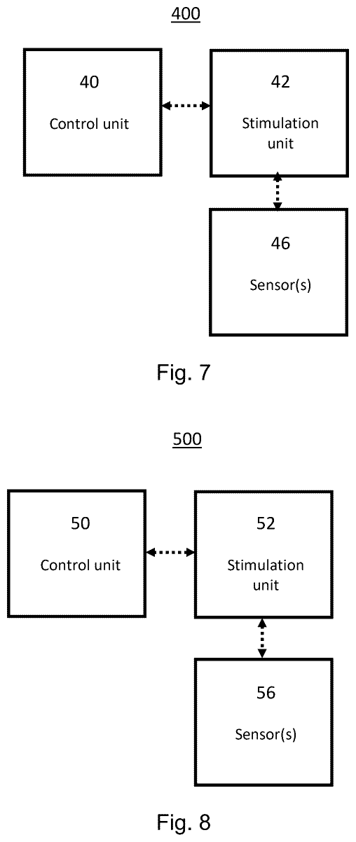

FIG. 7 provides a schematic overview of a neuromodulation/neurostimulation system 400 according to further embodiment of the present invention.

Here, the system 400 is a closed-loop system for supporting and/or controlling a coughing function in a patient with SCI and/or other neurological disorders.

The system 400 comprises at least one sensor 46, operatively connected to the at least one control unit 40.

For instance, the system 400 may include a single sensor 46.

The at least one sensor 46 is configured and/or adapted to detect a transition from the inhalation phase to the compressive phase, and from the compressive phase to the expulsive phase.

Also, the sensor 46 is configured an adapted to transmit data regarding a detected phase transition to the at least one control unit 40, in real time.

Based on the data received from the at least one sensor 46, the at least one control unit 40 is configured and/or adapted to control the at least one stimulation unit 42 to:
during the inhalation phase, deliver stimulation to control the diaphragm and/or the external intercostal muscles and/or the scalene muscles and/or the parasternal muscles, according to at least one predefined stimulation parameter for said inhalation phase; during the compressive phase, deliver stimulation to control the abdominal muscles and/or the oblique muscles and/or the internal intercostal muscles, according to at least one predefined stimulation parameter for said compressive phase, and
during the expulsive phase, deliver stimulation to control the abdominal muscles and/or the oblique muscles and/or the internal intercostal muscles, according to at least one predefined stimulation parameter for said expulsive phase.

FIG. 8 provides a schematic overview of a neuromodulation/neurostimulation system 500 according to a still further embodiment of the present invention.

Here, the system 500 is also a closed-loop system for supporting and/or controlling a coughing function in a patient with SCI and/or other neurological disorders.

The system 500 comprises at least one sensor 56, operatively connected to the at least one control unit 50.

For instance, the system 500 may include a single sensor 56.

The at least one sensor 56 is configured and/or adapted to detect a transition from the inhalation phase to the compressive phase, and from the compressive phase to the expulsive phase.

Also, the at least one sensor 56 is configured and/or adapted to transmit data regarding a detected phase transition to the at least one control unit 50, in real time.

Based on the data received from the at least one sensor 56, the at least one control unit 50 is configured and/or adapted to control the at least one stimulation unit 52 to:
during the inhalation phase, deliver stimulation to the cervical region of the spinal cord of the patient according to at least one predefined stimulation parameter for said inhalation phase, to control diaphragm activity, and
during the compressive phase and the expulsive phase, deliver stimulation to the thoracic region of the spinal cord of the patient according to at least one predefined stimulation parameter for said compressive phase and at least one predefined stimulation parameter for said expulsive phase, to control compression and expulsion.

Sensors that can be implemented with the neuromodulation/neurostimulation system 200, 400, 500 according to the invention may include one or more among:
a spirometer device;
a transcutaneous electromyography (EMG) sensor;
an implantable EMG sensor;
a photoplethysmography (PPG) sensor, and/or
a wearable strain sensor.

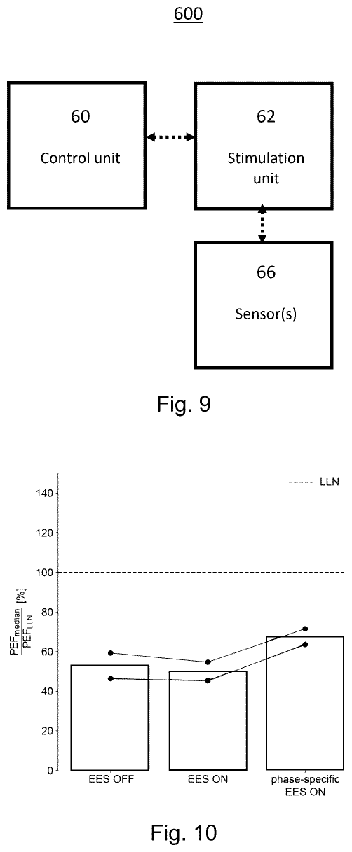

FIG. 9 provides a schematic overview of a neuromodulation/neurostimulation system 600 according to still another embodiment of the present invention.

Here, the system 600 is a closed-loop system for supporting and/or controlling a breathing function in a patient with SCI and/or other neurological disorders.

The system 600 comprises at least one sensor 66, operatively connected to the at least one control unit 60.

For instance, the system may comprise a single sensor 66.

The at least one sensor 66 is configured and/or adapted to detect a diaphragm activity of the patient.

In particular, the at least one sensor 66 includes at least one implantable EEG sensor.

For instance, the at least one sensor 66 may include a single implantable EEG sensor.

Also, the at least one sensor 66 is configured and/or adapted to transmit data on the detected diaphragm activity to the at least one control unit 60, in real time.

The control unit 60 is configured and/or adapted to control the at least one stimulation unit 62 to deliver stimulation based on the detected diaphragm activity of the patient.

FIG. 12 illustrates an example of a stimulation signal, optionally generatable and/or deliverable by or in any of the preceding embodiments. Referring to FIG. 12 (a), the signal is represented schematically as a sequence of stimulation pulses 80. Each pulse is represented schematically as a single line, although it is to be understood that the actual pulses may have a different mark: space ratio from that illustrated. Also, it is to be understood that each pulse may be an envelope within which plural pulses of a higher frequency carrier (not shown) wave may be generated. The pulses may be monophasic, or biphasic.

In the current example, the frequency of the pulses 80 is illustrative of one or more stimulation parameters or characteristics that can be varied between a first phase 82, and a second phase 84, of the respiration cycle. The first phase 82 may be an inhalation phase, and the second phase 84 may be an expiration phase, although the two may also be swapped in the current example. FIG. 12 (b) represents schematically the frequency of the pulses. During the first phase 82, the pulses have a first frequency F1, and during the second phase 84, the pulses have a second frequency F2. In the current example, the second frequency F2 is higher than the first frequency F1. Additionally or alternatively, the frequency switches at the transition from one phase 82, 84 to another. In other examples, the frequency may transition more progressively between one phase 82, 84 and another. For example, the frequency may begin to transition progressively before the end of one phase 82, 84. The progression may be generally linear, or curvilinear, or non-linear. FIG. 12 (c) represents schematically that the frequency may optionally vary generally continuously between the first frequency value F1 in the first phase 82 and the second frequency value F2 in the second phase 84.

The first frequency F1 (e.g. during an inhalation phase) may, in some cases, be within a range of 100 to 200 Hz, optionally about 120 Hz. The second frequency F2 (e.g. during an expiration phase) may, in some cases, be within a range of about 150 Hz to 400 Hz, preferably in a range of 200 Hz to 400 Hz, optionally about 250 Hz.

The second frequency F2 may generally about twice the value of the first frequency F1.

It is believed that such stimulation parameters can greatly assist activation of the respective muscles during the different phases of the respiration function of a patient, whether the stimulation is applied at different respective regions of the spinal cord (according to the respective phase), or even if the stimulation is applied at the same region of the spinal cord during two or more phases. The stimulation can follow a patient's natural respiration function (for example, but not exclusively, if generated by closed-loop control), or the stimulation can guide and/or control a patient's natural respiration (for example, but not exclusively, if generated without closed-loop control).

Additionally or alternatively to frequency, one or more other stimulation parameters may be controlled to vary depending on the respective phase of the respiration function, for example, any of stimulation amplitude, pulse width, carrier frequency.

The neuromodulation/neurostimulation system 100, 200, 300, 400, 500, 600 according to the invention is suitable for use in a method for supporting and/or controlling a function of the respiratory system, e.g. breathing or coughing, in a patient with SCI and/or other neurological disorders.

Note that the example control and estimation routines included herein can be used with various neuromodulation and/or neurostimulation system configurations. The control methods and routines disclosed herein may be stored as executable instructions in non-transitory memory and may be carried out by a control unit such as a microcontroller (or a computer) in combination with the at least one control unit 10, 20, 30, 40, 50, 60, the at least one stimulation unit 12, 22, 32, 42, 52, 62, the feedback module and/or the at least one sensor 26, 46, 56, 66. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example embodiments described herein but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of a computer readable storage medium in a control unit (e.g. a microcontroller) of the system, where the described actions are carried out by executing the instructions in a system including the various hardware components in combination with an electronic control unit.

### Clinical Trial on Human SCI Patients

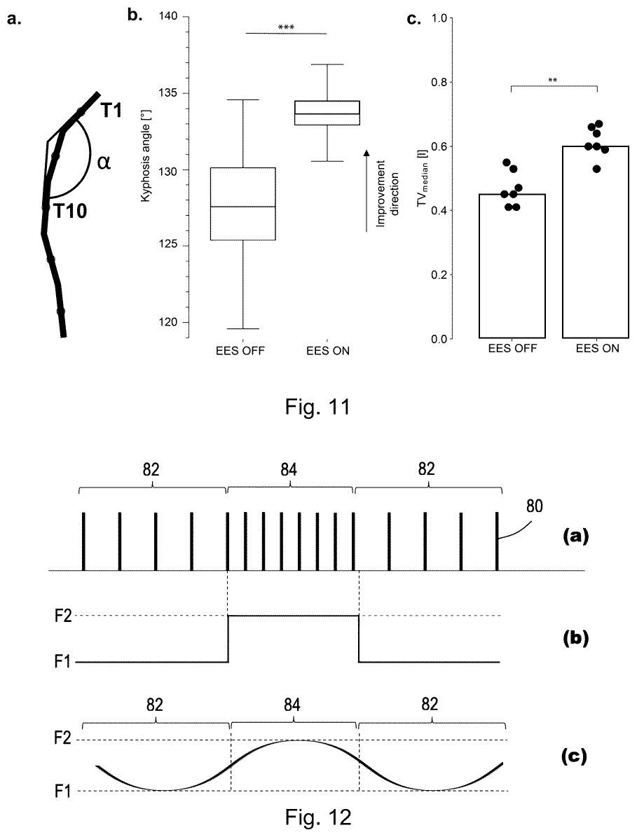

FIGS. 10-11 show diagrams referring to a clinical trial on human patients having SCI affecting a function of the respiratory system, in particular coughing.

All the involved patients had a peak expiratory flow (PEF) that was below the Lower Limit of Normality (LLN) in the absence of EES (FIG. 10).

In particular, the LLN was calculated based on ERS (ECCS)/Knudson standards.

The clinical trial revealed that delivery of phase-specific EES has a positive effect on PEF, thereby providing an effective coughing support (FIG. 10).

FIG. 11a provides a schematic representation of a spine curvature reconstruction from inertial measurement unit (IMUs) data.

The α angle represents the kyphosis angle.

FIG. 11b shows the effect of EES on the kyphosis angle in one study participant ***p-value<0.001, two-sample t-test).

In particular, a larger kyphosis angle represents a more upright posture of the spinal cord, which is beneficial in order to relieve resistance to the diaphragm and the rib cage, allowing the patient to perform a function of the respiratory system such as coughing with increased effectiveness and reduced effort.

FIG. 11c shows tidal volume (TV) improvement when EES is applied in one study participant (**p-value=0,0022, Mann-Whitney U test) .

These results suggest that phase-specific EES to the dorsal roots innervating the trunk muscles provides a more upright posture and an increased tidal volume.

### REFERENCES

100, 200, 300, 400, 500, 600 Neuromodulation/neurostimulation system
10, 20, 30, 40, 50, 60 Control unit
12, 22, 32, 42, 52, 62 Stimulation unit
14,34 Feedback module
26, 46, 56, 66 Sensor(s)
80 Stimulation pulses
82,84 Phases of respiration function
F1, F2 Stimulation frequencies

1-30. (canceled)

31. A neuromodulation/neurostimulation system for supporting and/or controlling a function of a respiratory system of a patient, the function comprising a plurality of phases each involving one or more muscles, the system comprising:
at least one control unit, configured to provide stimulation data for at least a first phase and a second phase of the function of the respiratory system, and
at least one stimulation unit, operatively connected to the at least one control unit,
wherein the at least one stimulation unit is configured to deliver electrical stimulation at least partially targeted to dorsal roots of a thoracic region of a spinal cord innervating trunk muscles of the patient,
wherein the stimulation data includes at least one stimulation parameter defined for each of the first phase and the second phase of the function of the respiratory system, and
wherein, during at least the first phase and the second phase of the function of the respiratory system, the at least one control unit is configured to control the at least one stimulation unit to deliver the electrical stimulation according to the at least one stimulation parameter defined for the ongoing phase of the function.

32. The neuromodulation/neurostimulation system of claim 31, wherein the at least one stimulation unit is configured to deliver an electrical stimulation targeted to dorsal roots of a low thoracic region or an upper thoracic region of the spinal cord of the patient.

33. The neuromodulation/neurostimulation system of claim 31, wherein the at least one stimulation unit is further configured to deliver an electrical stimulation targeted to dorsal roots of a cervical region of the spinal cord of said patient.

34. The neuromodulation/neurostimulation system of claim 31, wherein the at least one stimulation unit is configured to deliver electrical stimulation targeted to dorsal roots at the spinal cord level of:
C2-C4 for controlling the diaphragm,
T1-T12 for controlling the intercostal muscles, or
T7-L1 for controlling the abdominal and oblique muscles.

35. The neuromodulation/neurostimulation system of claim 31, wherein the at least one stimulation unit is configured to deliver Epidural Electrical Stimulation (EES).

36. The neuromodulation/neurostimulation system of claim 31, wherein the at least one stimulation unit is configured to deliver Transcutaneous Spinal Cord Stimulation (tSCS).

37. The neuromodulation/neurostimulation system of claim 31, wherein the at least one stimulation unit is configured to deliver Epidural Electrical Stimulation (EES) in combination with at least one of:
a Transcutaneous Spinal Cord Stimulation (tSCS);
a Functional Electrical Stimulation (FES);
a Peripheric Nerve Stimulation (PNS);
a Vagus Nerve Stimulation (VNS);
a Neuromuscular Electrical Stimulation (NMES), and
a Diaphragm pacing/pacemakers.

38. The neuromodulation/neurostimulation system of claim 31, wherein the system is an open-loop system, the system further comprises a feedback module being configured to provide a feedback to the patient on a transition from one phase to a subsequent phase of the function of the respiratory system, such that the patient synchronizes his/her actions according to the received feedback from the feedback module.

39. The neuromodulation/neurostimulation system of claim 31, wherein the system is a closed-loop system, the system further comprises at least one sensor operatively connected to the at least one control unit, wherein:
the at least one sensor is configured to detect a transition from one phase to a subsequent phase of the function of the respiratory system, and transmit data regarding a detected phase transition to the at least one control unit in real time, and
based on the data received from the at least one sensor, the at least one control unit is configured to control the at least one stimulation unit to deliver targeted stimulation for controlling one or more muscles involved for the ongoing phase of the function of the respiratory system, according to at least one predefined stimulation parameter for the ongoing phase.

40. The neuromodulation/neurostimulation system of claim 31, wherein the function of the respiratory system is a coughing function, including:
an inhalation phase;
a compressive phase, and
an expulsive phase.

41. The neuromodulation/neurostimulation system of claim 40, wherein the system is an open-loop system, the system further comprises a feedback module, the feedback module being configured to provide a feedback to the patient on a transition from the inhalation phase to the compressive phase, and from the compressive phase to the expulsive phase, such that the patient synchronizes his/her actions based on the received feedback from the feedback module.

42. The neuromodulation/neurostimulation system of claim 40, wherein the system is a closed-loop system, the system further comprises at least one sensor operatively connected to the at least one control unit, wherein:
the at least one sensor is configured to detect a transition from the inhalation phase to the compressive phase, and from the compressive phase to the expulsive phase, and transmit data regarding the detected phase transition to the at least one control unit in real time, and
based on the data received from the at least one sensor, the at least one control unit is configured to control the at least one stimulation unit to:
during the inhalation phase, deliver stimulation to control a diaphragm, external intercostal muscles, scalene muscles, or parasternal muscles, according to at least one predefined stimulation parameter for the inhalation phase;
during the compressive phase, deliver stimulation to control abdominal muscles, oblique muscles, or internal intercostal muscles, according to at least one predefined stimulation parameter for the compressive phase, and
during the expulsive phase, deliver stimulation to control the abdominal muscles, the oblique muscles, or the internal intercostal muscles, according to at least one predefined stimulation parameter for the expulsive phase.

43. The neuromodulation/neurostimulation system of claim 40, wherein the system is a closed-loop system, the system further comprises at least one sensor operatively connected to the at least one control unit, wherein:
the at least one sensor is configured to detect a transition from the inhalation phase to the compressive phase, and from the compressive phase to the expulsive phase, and transmit data regarding a detected phase transition to the at least one control unit in real time; and
based on the data received from the at least one sensor, the at least one control unit is configured to control the at least one stimulation unit to:
during the inhalation phase, deliver stimulation to a cervical region of the spinal cord of the patient according to at least one predefined stimulation parameter for the inhalation phase, to control diaphragm activities, and
during the compressive phase and the expulsive phase, deliver stimulation to a thoracic region of the spinal cord of the patient according to at least one predefined stimulation parameter for the compressive phase and at least one predefined stimulation parameter for the expulsive phase, to control compression and expulsion.

44. The neuromodulation/neurostimulation system of claim 39, wherein the at least one sensor comprises at least one of:
a spirometer device;
a transcutaneous electromyography (EMG) sensor;
an implantable EMG sensor;
a photoplethysmography (PPG) sensor, and
a wearable strain sensor.

45. The neuromodulation/neurostimulation system of claim 31, wherein the function of the respiratory system is a breathing function comprising an inhalation phase and an expiration phase.

46. The neuromodulation/neurostimulation system of claim 45, wherein the system is a closed-loop system, the system further comprises at least one sensor operatively connected to the at least one control unit, the at least one sensor being configured to detect a diaphragm activity of the patient and transmit data on the detected diaphragm activity to the at least one control unit in real time, wherein the at least one control unit is configured to control the at least one stimulation unit to deliver stimulation based on the detected diaphragm activity of the patient.

47. The neuromodulation/neurostimulation system of claim 46, wherein the at least one sensor comprises at least one implantable EEG sensor.

48. The neuromodulation/neurostimulation system of claim 31, wherein the at least one stimulation parameter comprises at least one of a stimulation frequency, a stimulation amplitude, a pulse width, and a carrier frequency.

49. The neuromodulation/neurostimulation system of claim 31, wherein the patient is a human affected by spinal cord injury (SCI) or neurological disorders of a stroke, multiple sclerosis, an autonomic failure, an autonomic neuropathy, or a cancer of neurological tissue.

50. The neuromodulation/neurostimulation system of claim 38, the system further comprises a mobile device comprising display means allowing the patient to provide a user input to trigger a function of the respiratory system based on the feedback from the feedback module.

51. The neuromodulation/neurostimulation system of claim 31, wherein the at least one control unit is further configured to control the at least one stimulation unit to deliver the electrical stimulation at least partially targeted to the dorsal roots of the thoracic region of the spinal cord innervating the trunk muscles for blood pressure control.

52. The neuromodulation/neurostimulation system of claim 31, wherein the at least one stimulation parameter comprises a parameter that is different during the first phase than during the second phase.

53. The neuromodulation/neurostimulation system of claim 31, wherein the at least one stimulation parameter comprises a parameter that switches value at a transition between the first phase and the second phase.

54. The neuromodulation/neurostimulation system of claim 31, wherein the at least one stimulation parameter comprises a parameter that transitions progressively between the first phase and the second phase.

55. The neuromodulation/neurostimulation system of claim 31, wherein the at least one parameter comprises a signal frequency that is different during the first phase and during the second phase.

56. The neuromodulation/neurostimulation system of claim 55, wherein the signal frequency during an inhalation phase is lower than the signal frequency during an expiration phase.

57. A method of operating a neuromodulation and/or neurostimulation system for supporting and/or controlling a function of a respiratory system of a patient, the function comprising a plurality of phases each involving one or more muscles, the method comprising:
generating, during or for a first phase of a respiratory cycle, a first stimulation signal having a first characteristic; and
generating, during or for a second phase of the respiratory cycle, a second stimulation signal having a second characteristic at least partially different from the first characteristic.

58. The method of neuromodulation and/or neurostimulation of claim 57, further comprising:
delivering a first electrical stimulation to a first region of a spinal cord during or for the first phase of the respiratory cycle, the first electrical stimulation having the first characteristic; and
delivering a second electrical stimulation to a second region of the spinal cord during or for the second phase of the respiratory cycle, the second electrical stimulation having the second characteristic at least partially different from the first characteristic.

59. The method of claim 57, wherein the difference between the first and second characteristics comprises one or more of:
a stimulation frequency;
a stimulation amplitude;
a pulse width; and
a carrier frequency.

60. The method of claim 27, 28 or 29, wherein the difference between the first and second characteristics comprises:
a progressive transition between one phase and another, or
a switch in a characteristic of the signal at a transition between one phase and another.
